(19) United States
(12) Patent Application Publication
(10) Pub. No.: US 2014/0163100 A1
(43) Pub. Date: Jun. 12, 2014

(54) Methods of Treating Multiple Sclerosis and Preserving and/or Increasing Myelin Content

(71) Applicant: BIOGEN IDEC MA INC., Cambridge, MA (US)

(72) Inventors: Katherine Dawson, Westwood, MA (US); Gilmore O'Neill, Medford, MA (US); Alfred Sandrock, Newton, MA (US)

(73) Assignee: Biogen IDEC MA Inc., Cambridge, MA (US)

(21) Appl. No.: 14/119,373

(22) PCT Filed: May 25, 2012
(86) PCT No.: PCT/US12/39721

§ 371 Date: Feb. 18, 2014

Related U.S. Application Data
(60) Provisional application No. 61/490,572, filed on May 26, 2011.
(60) Provisional application No. 61/625,624, filed on Apr. 17, 2012.

(51) Int. Cl. A61K 31/225

(52) U.S. Cl. CPC A61K 31/225
USPC 514/547

(57) ABSTRACT

Methods of treating multiple sclerosis in a subject, including: reducing the frequency of relapse, reducing the annualized relapse rate, reducing the risk of disability progression, reducing the number of new or newly enlarging T2 lesions, reducing the number of gadolinium lesions; and methods of preserving/increasing myelin content in a subject having multiple sclerosis; by daily administering a composition containing a fumarate, such as dimethyl fumarate or monomethyl fumarate, to the subject.

### BRIEF SUMMARY OF THE INVENTION

The methods provided are exemplary and are not intended to limit the scope of the claimed embodiments.

Provided are methods and compositions for treating a subject having multiple sclerosis. In one embodiment, the subject has multiple sclerosis, such as relapsing-remitting multiple sclerosis, and is administered about 480 mg per day of a fumarate (e.g., dimethyl fumarate, monomethyl fumarate, or a combination thereof) for a period of time sufficient to achieve one or more of the following changes: (a) reduced frequency of relapse in the subject; (b) reduced probability of relapse in the subject; (c) reduced annualized relapse rate in the subject; (d) reduced risk of disability progression in the subject; (e) reduced number of new or newly enlarging T2 lesions in the subject; (f) reduced number of new non-enhancing T1 hypointense lesions in the subject; and (g) reduced number of Gd+ lesions in the subject; wherein the changes (a)-(g) are relative to a subject treated with placebo.

In one embodiment, the subject has multiple sclerosis, such as relapsing-remitting multiple sclerosis, and is administered about 480 mg per day of a fumarate (e.g., dimethyl fumarate, monomethyl fumarate, or a combination thereof) for a period of time sufficient to achieve one or more of changes (a)-(e) and (g) listed above.

In one embodiment, the subject administered the fumarate (e.g., dimethyl fumarate, monomethyl fumarate, or a combination thereof) has not received any multiple sclerosis treatment before being treated with fumarate.

In another embodiment, a subject having relapsing-remitting multiple sclerosis is administered about 480 mg per day of a fumarate (e.g., dimethyl fumarate, monomethyl fumarate, or a combination thereof) for a period of time sufficient to achieve one or more of the following changes: (a) reduced annualized relapse rate of at least 30%; (b) reduced risk of disability progression of at least 30%; and (c) reduced number of new or newly enlarging T2 lesions of at least 65% in the subject, wherein the changes (a)-(c) are relative to a subject treated with placebo.

In another embodiment, a subject having relapsing-remitting multiple sclerosis is administered about 720 mg of a fumarate (e.g., dimethyl fumarate, monomethyl fumarate, or combinations thereof) per day for at least 24 weeks.

In another embodiment, a subject having multiple sclerosis is administered about 480 mg per day of a fumarate (e.g., dimethyl fumarate, monomethyl fumarate, or combinations thereof) for a period of time sufficient to preserve and/or increase myelin content in the subject.

In one embodiment, a subject younger than 40 years of age having multiple sclerosis is administered about 480 mg per day of a fumarate (e.g., dimethyl fumarate, monomethyl fumarate, or combinations thereof) for a period of time sufficient to achieve one or more of the following changes: (a) reduced frequency of relapse in the subject; (b) reduced probability of relapse in the subject; (c) reduced annualized relapse rate in the subject; (d) reduced risk of disability progression in the subject; (e) reduced number of new or newly enlarging T2 lesions in the subject; (f) reduced number of new non-enhancing T1 hypointense lesions in the subject; and (g) reduced number of Gd+ lesions in the subject; wherein the changes (a)-(g) are relative to a subject treated with placebo.

### BRIEF DESCRIPTION OF THE DRAWINGS

FIG. 1 reports the proportion of subjects relapsed to a pre-specified primary endpoints after a 96 week period in a first Phase 3 clinical trial in which subjects with relapsing-remitting multiple sclerosis were administered a placebo, dimethyl fumarate (BG-12) twice daily (BID) at 240 mg per dose, or dimethyl fumarate (BG-12) three times daily (TID) at 240 mg per dose.

FIG. 2 reports a distribution of relapses of subjects over a 2 year time period in a first Phase 3 clinical trial in which subjects with relapsing-remitting multiple sclerosis were administered a placebo, dimethyl fumarate (BG-12) twice daily (BID) at 240 mg per dose, or dimethyl fumarate (BG-12) three times daily (TID) at 240 mg per dose.

FIG. 3 reports a risk of relapse after a 60 week period in a first Phase 3 clinical trial in which subjects with relapsing-remitting multiple sclerosis were administered a placebo or dimethyl fumarate (BG-12).

FIG. 4 reports a progression of disability after a 96 week period in a first Phase 3 clinical trial in which subjects with relapsing-remitting multiple sclerosis were administered a placebo, dimethyl fumarate (BG-12)) twice daily (BID) at 240 mg per dose, or dimethyl fumarate (BG-12) three times daily (TID) at 240 mg per dose.

FIG. 5 reports a distribution of new or newly enlarging T2 lesions after a 2 year time period in a first Phase 3 clinical trial in which subjects with relapsing-remitting multiple sclerosis were administered a placebo, dimethyl fumarate (BG-12) twice daily (BID) at 240 mg per dose, or dimethyl fumarate (BG-12) three times daily (TID) at 240 mg per dose.

FIG. 6 reports a distribution of new Gd+ lesions observed in subjects in a first Phase 3 clinical trial conducted over a 2 year time period in which subjects with relapsing-remitting multiple sclerosis were administered a placebo, dimethyl fumarate (BG-12) twice daily (BID) at 240 mg per dose, or dimethyl fumarate (BG-12) three times daily (TID) at 240 mg per dose.

FIG. 7 depicts a mean change in magnetization transfer ratio (“MTR”) from baseline in whole brain (“WB”) observed in subjects in a first Phase 3 clinical trial conducted over a 2 year time period in which subjects with relapsing-remitting multiple sclerosis were administered a placebo, dimethyl fumarate (BG-12) twice daily (BID) at 240 mg per dose, or dimethyl fumarate (BG-12) three times daily (TID) at 240 mg per dose.

FIG. 8 depicts a mean change in magnetization transfer ratio (“MTR”) from baseline in normal appearing brain tissue (“NABT”) observed in subjects in a first Phase 3 clinical trial conducted over a 2 year time period in which subjects with relapsing-remitting multiple sclerosis were administered a placebo, dimethyl fumarate (BG-12) twice daily (BID) at 240 mg per dose, or dimethyl fumarate (BG-12) three times daily (TID) at 240 mg per dose.

FIG. 9 reports an annualized relapse rate observed in subjects in a second Phase 3 clinical trial conducted over a 2 year time period in which subjects with relapsing-remitting multiple sclerosis were administered a placebo, dimethyl fumarate (BG-12) twice daily (BID) at 240 mg per dose, dimethyl fumarate (BG-12) three times daily (TID) at 240 mg per dose, or 20 mg glatiramer acetate once daily.

FIG. 10 reports a distribution of relapses observed in subjects in a second Phase 3 clinical trial conducted over a 2 year time period in which subjects with relapsing-remitting multiple sclerosis were administered a placebo, dimethyl fumarate (BG-12) twice daily (BID) at 240 mg per dose, dimethyl fumarate (BG-12) three times daily (TID) at 240 mg per dose, or 20 mg glatiramer acetate once daily.

FIG. 11 reports the proportion of subjects relapsed to a pre-specified primary endpoints after a 96 week period in a second Phase 3 clinical trial in which subjects with relapsing-remitting multiple sclerosis were administered a placebo, dimethyl fumarate (BG-12) twice daily (BID) at 240 mg per dose, dimethyl fumarate (BG-12) three times daily (TID) at 240 mg per dose, or 20 mg glatiramer acetate once daily.

FIG. 12 reports the time to 12-week confirmed disability progression in a second Phase 3 clinical trial in which subjects with relapsing-remitting multiple sclerosis were administered a placebo, dimethyl fumarate (BG-12) twice daily (BID) at 240 mg per dose, dimethyl fumarate (BG-12) three times daily (TID) at 240 mg per dose, or 20 mg glatiramer acetate once daily.

FIG. 13A reports a distribution of new or newly enlarging T2 lesions after a 2 year time period in a second Phase 3 clinical trial in which subjects with relapsing-remitting multiple sclerosis were administered a placebo, dimethyl fumarate (BG-12) twice daily (BID) at 240 mg per dose, dimethyl fumarate (BG-12) three times daily (TID) at 240 mg per dose, or 20 mg glatiramer acetate once daily.

FIG. 13B a distribution of new T1 hypointense lesions after a 2 year time period in a second Phase 3 clinical trial in which subjects with relapsing-remitting multiple sclerosis were administered a placebo, dimethyl fumarate (BG-12) twice daily (BID) at 240 mg per dose, dimethyl fumarate (BG-12) three times daily (TID) at 240 mg per dose, or 20 mg glatiramer acetate once daily.

FIG. 14 reports a mean number of Gd+ lesions observed in subjects in a second Phase 3 clinical trial conducted over a 2 year time period in which subjects with relapsing-remitting multiple sclerosis were administered a placebo, dimethyl fumarate (BG-12) twice daily (BID) at 240 mg per dose, dimethyl fumarate (BG-12) three times daily (TID) at 240 mg per dose, or 20 mg glatiramer acetate once daily.

FIG. 15A reports the median percent change from baseline in T2 hyperintense lesion volume observed after 1 year in subjects of a second Phase 3 clinical trial in which the subjects were administered a placebo, dimethyl fumarate (BG-12) twice daily (BID) at 240 mg per dose, dimethyl fumarate (BG-12) three times daily (TID) at 240 mg per dose, or 20 mg glatiramer acetate once daily.

FIG. 15B reports the median percent change from baseline in T2 hyperintense lesion volume observed after 2 years in subjects of a second Phase 3 clinical trial in which the subjects were administered a placebo, dimethyl fumarate (BG-12) twice daily (BID) at 240 mg per dose, dimethyl fumarate (BG-12) three times daily (TID) at 240 mg per dose, or 20 mg glatiramer acetate once daily.

FIG. 16A reports the median percent change from baseline in T1 hypointense lesion volume observed after 1 year in subjects of a second Phase 3 clinical trial in which the subjects were administered a placebo, dimethyl fumarate (BG-12) twice daily (BID) at 240 mg per dose, dimethyl fumarate (BG-12) three times daily (TID) at 240 mg per dose, or 20 mg glatiramer acetate once daily.

FIG. 16B reports the median percent change from baseline in T1 hypointense lesion volume observed after 2 years in subjects of a second Phase 3 clinical trial in which the subjects were administered a placebo, dimethyl fumarate (BG-12) twice daily (BID) at 240 mg per dose, dimethyl fumarate (BG-12) three times daily (TID) at 240 mg per dose, or 20 mg glatiramer acetate once daily.

FIG. 17 reports the mean Gd⁺ lesion volume observed in subjects after 2 years in subjects of a second Phase 3 clinical trial in which the subjects were administered a placebo, dimethyl fumarate (BG-12) twice daily (BID) at 240 mg per dose, dimethyl fumarate (BG-12) three times daily (TID) at 240 mg per dose, or 20 mg glatiramer acetate once daily.

FIG. 18A reports the median percent change from baseline in whole brain volume observed after 2 years in subjects of a second Phase 3 clinical trial in which the subjects were administered a placebo, dimethyl fumarate (BG-12) twice daily (BID) at 240 mg per dose, dimethyl fumarate (BG-12) three times daily (TID) at 240 mg per dose, or 20 mg glatiramer acetate once daily.

FIG. 18B reports the median percent change from baseline in whole brain volume observed between week 24 and year 2 of a second Phase 3 clinical trial in which subjects were administered a placebo, dimethyl fumarate (BG-12) twice daily (BID) at 240 mg per dose, dimethyl fumarate (BG-12) three times daily (TID) at 240 mg per dose, or 20 mg glatiramer acetate once daily.

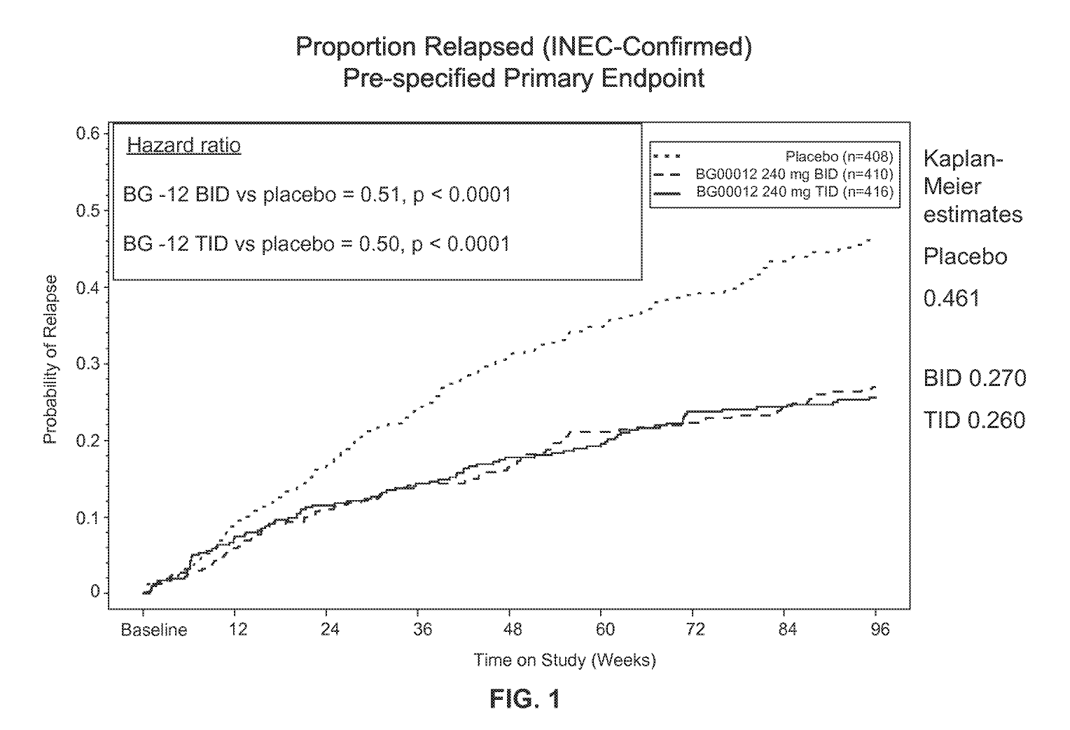

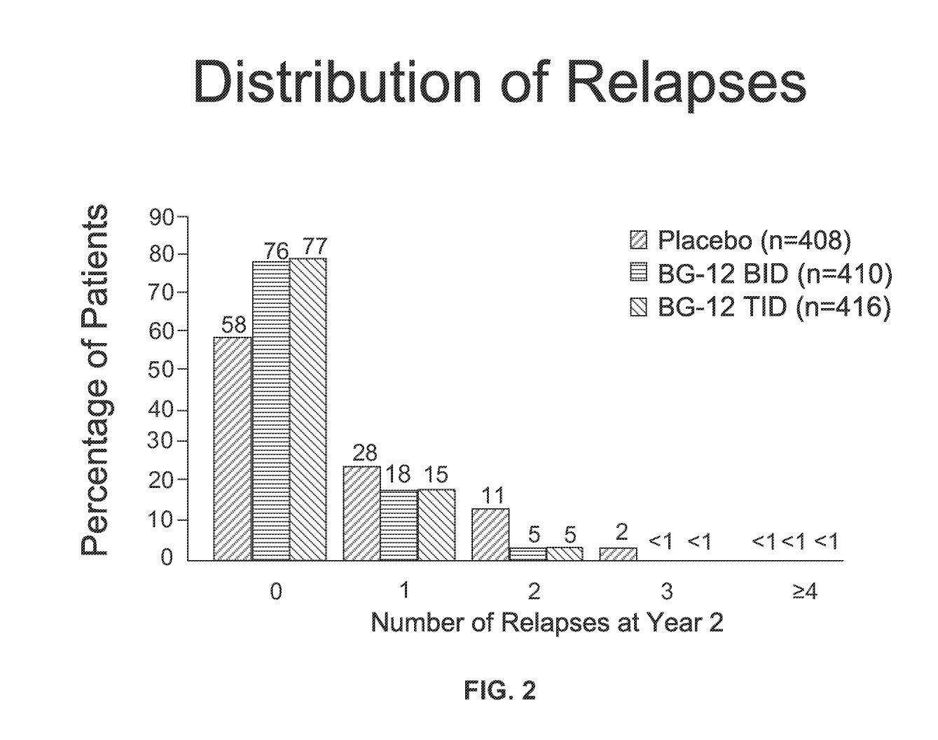

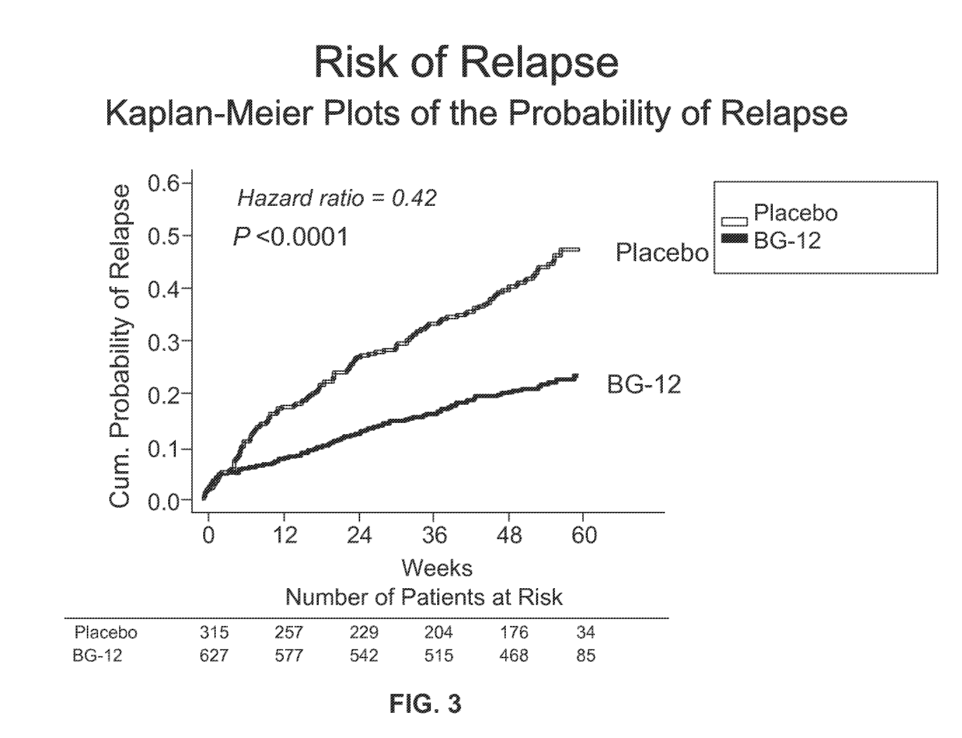

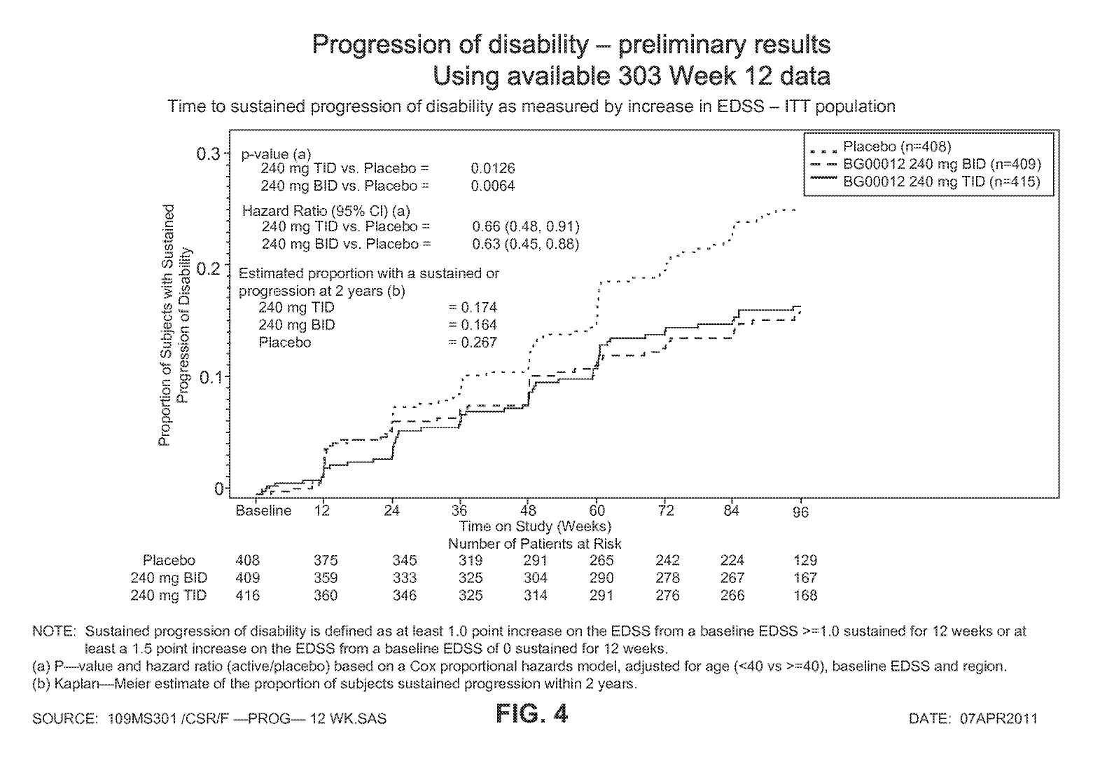

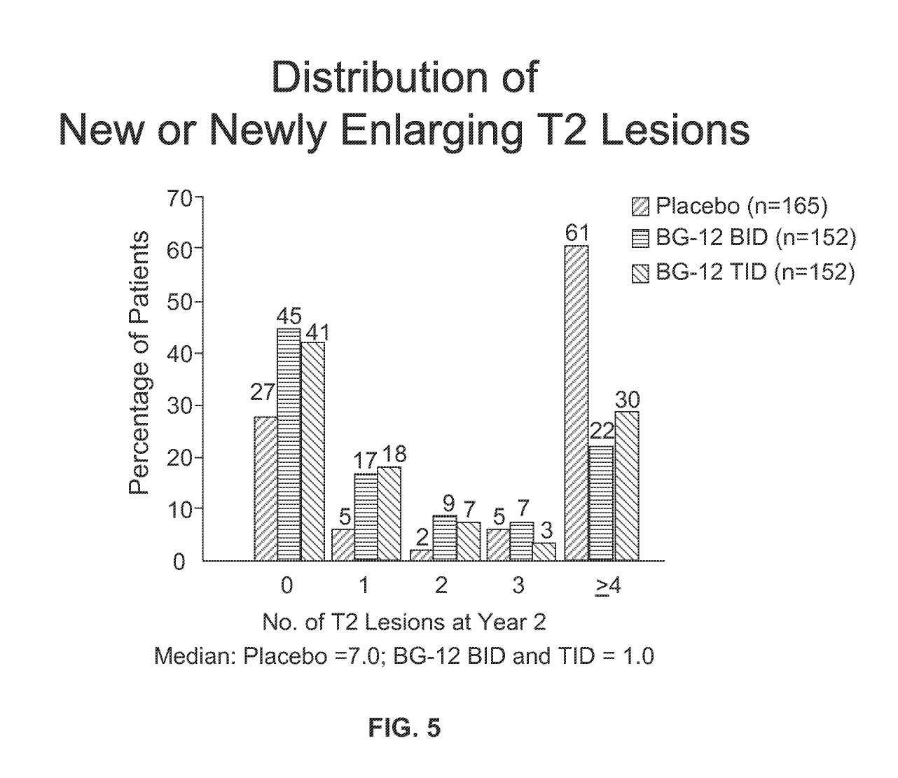

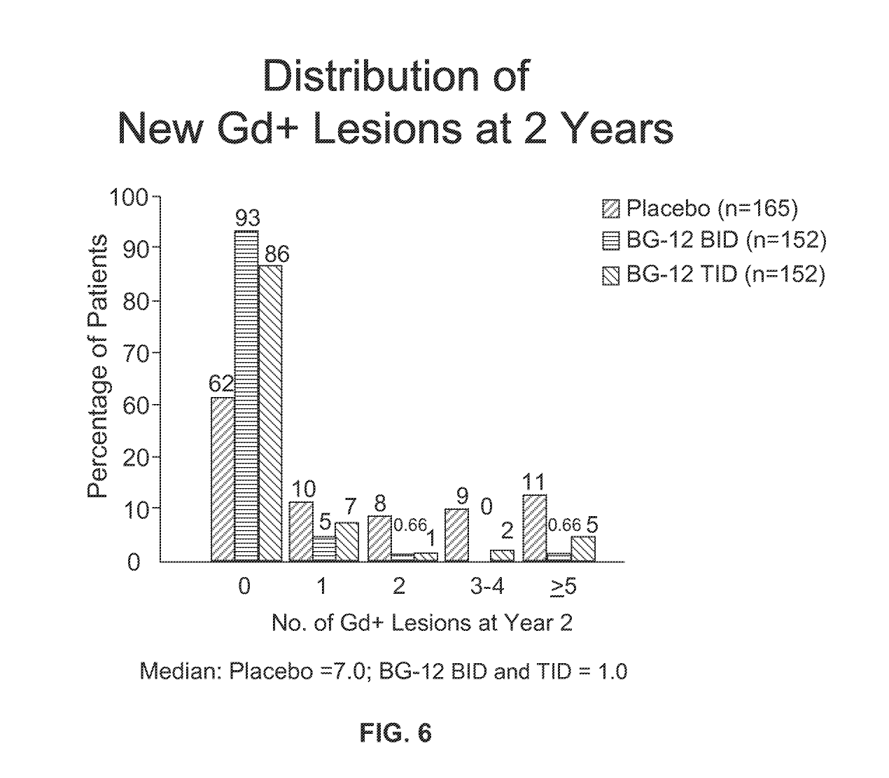

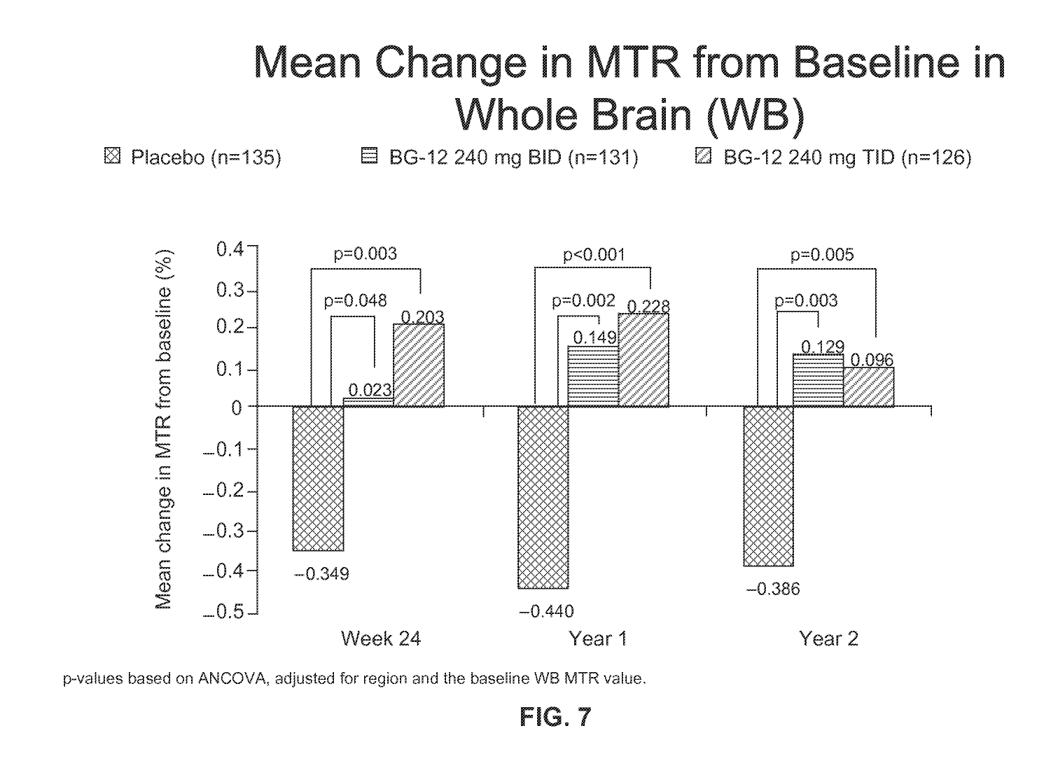

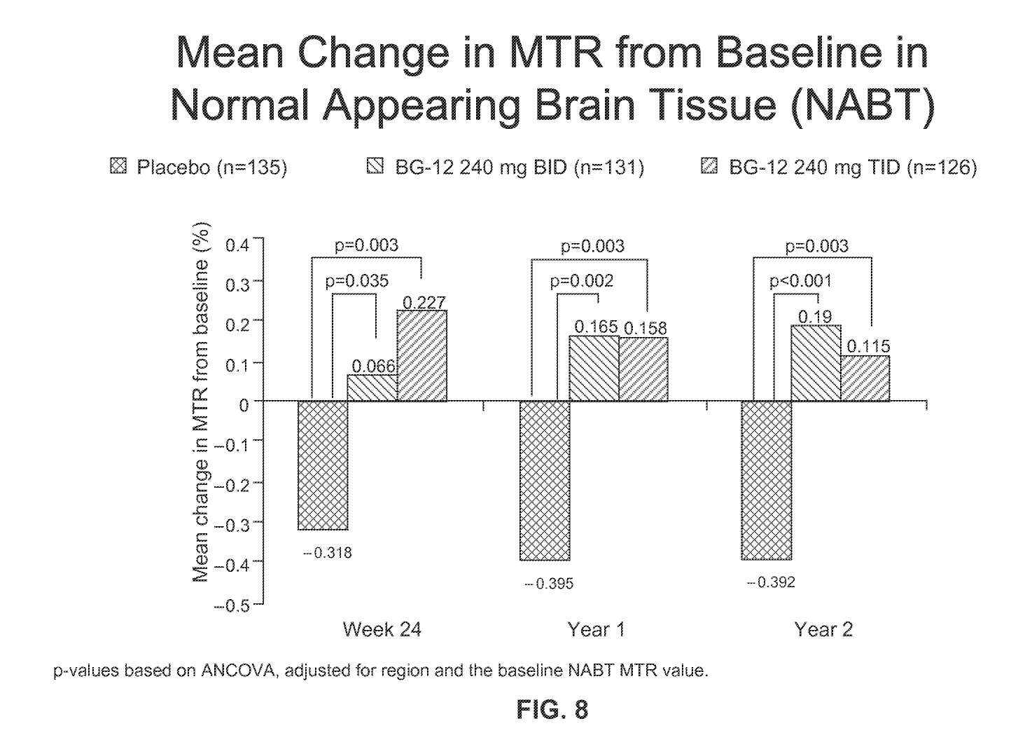

### DETAILED DESCRIPTION OF THE INVENTION

### Definitions

All of the various aspects, embodiments, and options disclosed herein can be combined in any and all variations.

As used herein, “a” means one or more unless otherwise specified.

Open terms such as “include,” “including,” “contain,” “containing” and the like mean “comprising.”

The term “treating” refers to administering a therapy in an amount, manner, or mode effective to improve a condition, symptom, or parameter associated with a disorder or to prevent progression of a disorder, to either a statistically significant degree or to a degree detectable to one skilled in the art. An effective amount, manner, or mode can vary depending on the subject and may be tailored to the subject. For neurological disorders referred herein, the treatments offered by the methods disclosed herein aim at improving the conditions (or lessening the detrimental effects) of the disorders and not necessarily at completely eliminating or curing the disorders.

Terms in this application control in the event of a conflict with a publication term that is incorporated by reference.

The term “or” can be conjunctive or disjunctive.

The term “placebo” refers to a composition without active agent (e.g., dimethyl fumarate, monomethyl fumarate, or combinations thereof). Placebo compositions can be prepared by known methods, including those described herein.

The term “EDSS” refers to an Expanded Disability Status Scale. The EDSS scale runs from 0 to 10 and is:
0 Normal neurological examination (all grade 0 in functional systems [FS]; cerebral grade 1 acceptable)
1 No disability, minimal signs in 1 FS (i.e. grade 1 excluding cerebral grade 1)
1.5 No disability, minimal signs in >1 FS (>1 grade 1 excluding cerebral grade 1)
2 Minimal disability in 1 FS (1 FS grade 2, others 0 or 1)
2.5 Minimal disability in 2 FS (2 FS grade 2, others 0 or 1)
3 Moderate disability in 1 FS (1 FS grade 3, others 0 or 1), or mild disability in 3-4 FS (3-4 FS grade 2, others 0 or 1) though fully ambulatory
3.5 Fully ambulatory but with moderate disability in 1 FS (1 FS grade 3) and 1-2 FS grade 2; or 2 FS grade 3; or 5 FS grade 2 (others 0 or 1)
4 Fully ambulatory without aid, self-sufficient, up and about some 12 hours a day despite relatively severe disability consisting of 1 FS grade 4 (others 0 or 1), or combinations of lesser grades exceeding limits of previous steps. Able to walk without aid or rest some 500 m
4.5 Fully ambulatory without aid, up and about much of the day, able to work a full day, may otherwise have some limitation of full activity or require minimal assistance; characterized by relatively severe disability, usually consisting of 1 FS grade 4 (others 0 or 1) or combinations of lesser grades exceeding limits of previous steps. Able to walk without aid or rest for some 300 m
5 Ambulatory without aid or rest for about 200 m; disability severe enough to impair full daily activities (e.g., to work full day without special provisions). (Usual FS equivalents are 1 grade 5 alone, others 0 or 1; or combination of lesser grades usually exceeding specifications for step 4.0)
5.5 Ambulatory without aid or rest for about 100 m, disability severe enough to preclude full daily activities. (Usual FS equivalents are 1 grade 5 alone, others 0 or 1; or combination of lesser grades usually exceeding those for step 4.0)
6 Intermittent or unilateral constant assistance (cane, crutch or brace) required to walk about 100 m with or without resting. (Usual FS equivalents are combinations with >2 FS grade 3+)
6.5 Constant bilateral assistance (canes, crutches or braces) required to walk about 20 m without resting. (Usual FS equivalents are combinations with >2 FS grade 3+)
7 Unable to walk beyond about 5 m even with aid, essentially restricted to wheelchair; wheels self in standard wheelchair and transfers alone; up and about in wheelchair some 12 hours a day. (Usual FS equivalents are combinations with >1 FS grade 4+; very rarely, pyramidal grade 5 alone)
7.5 Unable to take more than a few steps; restricted to wheelchair, may need aid in transfer; wheels self but cannot carry on in standard wheelchair a full day; may require motorized wheelchair. (Usual FS equivalents are combinations with >1 FS grade 4+)
8 Essentially restricted to bed or chair or perambulated in wheelchair, but may be out of bed itself much of the day; retains many self-care functions; generally has effective use of arms. (Usual FS equivalents are combinations, generally 4+ in several systems)
8.5 Essentially restricted to bed much of the day; has some effective use of arm(s); retains some self-care functions. (Usual FS equivalents are combinations, generally 4+ in several systems)
9 Helpless bedridden patient; can communicate and eat. (Usual FS equivalents are combinations, mostly grade 4+)
9.5 Totally helpless bedridden patient; unable to communicate effectively or eat/swallow. (Usual FS equivalents are combinations, almost all grade 4+)
10 Death due to multiple sclerosis

Discussion

Multiple sclerosis (“MS”) is an autoimmune disease with the autoimmune activity directed against central nervous system (“CNS”) antigens. The disease is characterized by inflammation in parts of the CNS, leading to the loss of the myelin sheathing around neuronal axons (demyelination), axonal loss, and the eventual death of neurons, oligodendrocytes and glial cells. For a comprehensive review of MS and current therapies, see e.g., McAlpine's Multiple Sclerosis, by Alastair Compston et al., 4th edition, Churchill Livingstone Elsevier, (2006).

An estimated 2,500,000 people in the world suffer from MS. It is one of the most common diseases of the CNS in young adults. MS is a chronic, progressing, disabling disease, which generally strikes its victims sometime after adolescence, with diagnosis generally made between 20 and 40 years of age, although onset may occur earlier. The disease is not directly hereditary, although genetic susceptibility plays a part in its development. MS is a complex disease with heterogeneous clinical, pathological and immunological phenotype.

There are four major clinical types of MS: 1) relapsing-remitting MS (“RR-MS”), characterized by clearly defined relapses with full recovery or with sequelae and residual deficit upon recovery; periods between disease relapses characterized by a lack of disease progression; 2) secondary progressive MS (“SP-MS”), characterized by initial relapsing remitting course followed by progression with or without occasional relapses, minor remissions, and plateaus; 3) primary progressive MS (“PP-MS”), characterized by disease progression from onset with occasional plateaus and temporary minor improvements allowed; and 4) progressive relapsing MS (“PR-MS”), characterized by progressive disease onset, with clear acute relapses, with or without full recovery; periods between relapses characterized by continuing progression.

Clinically, the illness most often presents as a relapsing-remitting disease and, to a lesser extent, as steady progression of neurological disability. Relapsing-remitting MS presents in the form of recurrent attacks of focal or multifocal neurologic dysfunction. Attacks may occur, remit, and recur, seemingly randomly over many years. Remission is often incomplete and as one attack follows another, a stepwise downward progression ensues with increasing permanent neurological deficit. The usual course of RR-MS is characterized by repeated relapses associated, for the majority of patients, with the eventual onset of disease progression. The subsequent course of the disease is unpredictable, although most patients with a relapsing-remitting disease will eventually develop secondary progressive disease. In the relapsing-remitting phase, relapses alternate with periods of clinical inactivity and may or may not be marked by sequelae depending on the presence of neurological deficits between episodes. Periods between relapses during the relapsing-remitting phase are clinically stable. On the other hand, patients with progressive MS exhibit a steady increase in deficits, as defined above and either from onset or after a period of episodes, but this designation does not preclude the further occurrence of new relapses.

MS pathology is, in part, reflected by the formation of focal inflammatory demyelinating lesions in the white matter, which are the hallmarks in patients with acute and relapsing disease. In patients with progressive disease, the brain is affected in a more global sense, with diffuse but widespread (mainly axonal) damage in the normal appearing white matter and demyelination also in the grey matter, particularly, in the cortex.

Most current therapies for MS are aimed at the reduction of inflammation and suppression or modulation of the immune system. As of 2006, the available treatments for MS reduce inflammation and the number of new episodes but not all of the treatments have an effect on disease progression. A number of clinical trials have shown that the suppression of inflammation in chronic MS rarely significantly limits the accumulation of disability through sustained disease progression, suggesting that neuronal damage and inflammation are independent pathologies. Thus, in advanced stages of MS, neurodegeneration appears to progress even in the absence of significant inflammation. Therefore, slowing demyelination, or promoting CNS remyelination as a repair mechanism, or otherwise preventing axonal loss and neuronal death are some of the important goals for the treatment of MS, especially, in the case of progressive forms of MS such as SP-MS.

Fumaric acid esters, such as dimethyl fumarate (“DMF”), have been previously proposed for the treatment of MS (see, e.g., Schimrigk et al., Eur. J. Neurol., 2006, 13(6):604-10; Drugs R&D, 2005, 6(4):229-30; U.S. Pat. No. 6,436,992).

DMF and monomethyl fumarate (“MMF”) can exert neuroprotective effects such as reduction in demyelination and axonal damage in a mouse MS model with characteristic features of advanced stages of chronic forms of MS. Although many well characterized rodent and primate models for MS exist, only recently have the characteristic features of progressive MS been identified in select animal models. Under the conditions tested, the neuroprotective effects of DMF and MMF appeared to be independent of their effect, if any, on inflammation, suggesting that use of these compounds may be advantageous in treating pathologies that exhibit progressive neurodegeneration even in the absence of a substantial inflammatory component.

Provided is a method of treating a subject with multiple sclerosis comprising administering to the subject a fumarate (e.g., DMF, MMF, or combinations thereof). One embodiment includes reducing frequency of relapse in the subject; or reducing the cumulative probability of relapse in the subject; or reducing the annualized relapse rate in the subject; or reducing the risk of disability progression in the subject; or reducing the number of new or newly enlarging T2 lesions in the subject; or reduced number of new non-enhancing T1 hypointense lesions in the subject; or reducing the number of Gd+ lesions in the subject, wherein the reductions are relative to a subject treated with placebo comprising administering to the subject a fumarate (e.g., DMF, MMF, or combinations thereof). In one embodiment, the subject being treated with the fumarate (e.g., DMF, MMF, or combinations thereof) has not received any multiple sclerosis treatment before being treated with the fumarate. In another embodiment, the subject being treated with the fumarate is less than 40 years of age.

Also provided is a method of preserving and/or increasing myelin content in a subject having multiple sclerosis comprising administering about 480 mg per day of a fumarate (e.g., DMF, MMF, or combinations thereof) to the subject for a period of time sufficient to preserve and/or increase the myelin content.

“Preserving and/or increasing the myelin content” means the content level of myelin in a group of subjects treated with fumarates (e.g., DMF, MMF, or a combination thereof) is on average higher that the content level of myelin in a placebo group of subjects. Fumarates (e.g., DMF) can reduce the rate of loss of myelin in a subject and/or lead to myelin regeneration. Either mechanism can lead to the results observed in Example 5.

In one embodiment, the myelin content of a group of subjects treated with DMF is preserved and/or increased relative to that of a group of subjects receiving placebo with a mean improvement of 0.5% and can be as much as about 1%, based on whole brain magnetization transfer ratio (MTR), after a prolonged period of treatment (e.g., 1 year, 2 years, 5 years, or longer).

The fumarate can be, for example, a compound that converts to methyl hydrogen fumarate in vivo after administration. In one embodiment, only some of the fumarate present in a pharmaceutical composition is converted to methyl hydrogen fumarate in vivo. In one embodiment, the fumarate is dimethyl fumarate, monomethyl fumarate, fumaric acid, a salt of monomethyl fumarate, a salt of fumaric acid, or any combination thereof. In another embodiment, the fumarate can be a compound of formula (I):

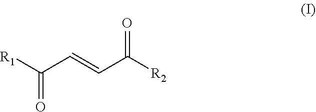

wherein R₁ and R₂ are independently OH, O⁻, C₁-C₆ alkoxy, or a pharmaceutically acceptable salt thereof. The C₁-C₆ alkoxy can be chosen from, for example, C₁-C₅ alkoxy, C₁-C₄ alkoxy, C₁-C₃ alkoxy, C₁-C₂ alkoxy, C₂-C₃ alkoxy, C₂-C₄ alkoxy, C₂-C₅ alkoxy, or C₂-C₆ alkoxy and may be linear or branched. In still yet another embodiment, the fumarate is a dialkyl fumarate.

In one embodiment, the methods described above comprise administering DMF. DMF has the structure:

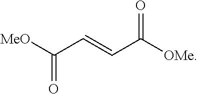

In some embodiments, the pharmaceutically acceptable salt can be a salt of a metal cation. The metal in the metal cation can be an alkali, alkaline earth, or transition metal such as Li, Na, K, Ca, Zn, Sr, Mg, Fe, or Mn.

Some inventive embodiments contemplate numerical ranges. Every numerical range provided herein includes the range endpoints as individual inventive embodiments. When a numerical range is provided, all individual values and subranges therein are present as if explicitly written out.

In some embodiments, any of the methods described above includes treating a subject with a fumarate (e.g., DMF, MMF, or combinations thereof). In some embodiments of any of the methods described above, the fumarate (e.g., DMF, MMF, or combinations thereof) can be administered in an amount ranging from about 1 mg/kg to about 50 mg/kg (e.g., from about 2.5 mg/kg to about 20 mg/kg or from about 2.5 mg/kg to about 15 mg/kg). The amount of fumarate (e.g., DMF, MMF, or combinations thereof) administered will also vary, as recognized by those skilled in the art, dependent on route of administration, excipient usage, and the possibility of co-usage with other therapeutic treatments including use of other therapeutic agents.

For example, a fumarate (e.g., DMF, MMF, or combinations thereof) can be administered to a subject, for example orally, in an amount of from about 0.1 g to about 1 g per day, or for example, in an amount of from about 100 mg to about 800 mg per day. The fumarate (e.g., DMF, MMF, or combinations thereof) can be administered, for example, in an amount of from about 120 mg per day to about 240 mg per day, from about 120 mg per day to about 480 mg per day; or from about 120 mg per day to about 720 mg per day.

For example, 720 mg of fumarate (e.g., DMF, MMF, or combinations thereof) per day may be administered in separate administrations of 2, 3, 4, 5 or 6 equal doses. For example, 480 mg of fumarate (e.g., DMF, MMF, or combinations thereof) per day may be administered as a single daily dose of 480 mg or as 2 daily dosages of 240 mg each. If the 480 mg of fumarate (e.g., DMF, MMF, or combinations thereof) is administered in 2 daily doses, each dose can consist of 2 tablets containing 120 mg of the fumarate for a total dose of 240 mg of the fumarate.

When two doses are administered per day, the time interval between administration of the first dose and the second dose can be, for example, about 8 hours apart, about 9 hours apart, about 10 hours apart, about 11 hours apart, or about 12 hours apart.

The fumarate (e.g., DMF, MMF, or combinations thereof) can be administered, for example, once daily in an amount of about 480 mg. The fumarate (e.g., DMF, MMF, or combinations thereof) can be administered once daily in an amount of 480 mg. The fumarate (e.g., DMF, MMF, or combinations thereof) can be daily administered in an amount ranging from about 400 mg to about 600 mg, about 410 mg to about 590 mg, about 420 mg to about 580 mg, about 430 mg to about 570 mg, about 440 mg to about 560 mg, about 450 mg to about 550 mg, about 460 mg to about 540 mg, about 470 mg to about 530 mg, about 480 mg to about 520 mg, or about 490 mg to about 510 mg. The fumarate (e.g., DMF, MMF, or combinations thereof) can be administered daily in an amount ranging from about 432 mg to about 528 mg. The fumarate (e.g., DMF, MMF, or combinations thereof) dose can be, for example: 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, or 490 mg.

The fumarate (e.g., DMF, MMF, or combinations thereof) can be administered in the form of a sustained or controlled release pharmaceutical formulation. Such formulation can be prepared by various technologies by a skilled person in the art. For example, the formulation can contain the fumarate (e.g., DMF, MMF, or combinations thereof), a rate-controlling polymer (i.e., a material controlling the rate at which the therapeutic compound is released from the dosage form) and optionally other excipients. Some examples of rate-controlling polymers are hydroxy alkyl cellulose, hydroxypropyl alkyl cellulose (e.g., hydroxypropyl methyl cellulose, hydroxypropyl ethyl cellulose, hydroxypropyl isopropyl cellulose, hydroxypropyl butyl cellulose and hydroxypropyl hexyl cellulose), poly(ethylene)oxide, alkyl cellulose (e.g., ethyl cellulose and methyl cellulose), carboxymethyl cellulose, hydrophilic cellulose derivatives, and polyethylene glycol. Compositions are described in WO 2006/037342.

The fumarate (e.g. DMF, MMF, or combinations thereof) can be administered by any method that permits the delivery of the fumarate (e.g., DMF, MMF, or combinations thereof) for treatment of multiple sclerosis, such as RR-MS. For instance, the fumarate (e.g., DMF, MMF, or combinations thereof) can be administered via pills, tablets, microtablets, pellets, micropellets, capsules (e.g., containing microtablets), suppositories, liquid formulations for oral administration, in the form of dietary supplements, nutritional supplements, or a food. The pharmaceutically acceptable compositions can include well-known pharmaceutically acceptable excipients, e.g., if the composition is an aqueous solution containing the active agent, it can be an isotonic saline, 5% glucose, or others. Solubilizing agents such as cyclodextrins, or other solubilizing agents well known to those familiar with the art, can be utilized as pharmaceutical excipients for delivery of the therapeutic compound. See, e.g., U.S. Pat. Nos. 6,509,376 and 6,436,992 for some formulations containing DMF or MMF. As to route of administration, the compositions can be administered orally, intranasally, transdermally, subcutaneously, intradermally, vaginally, intraaurally, intraocularly, intramuscularly, buccally, rectally, transmucosally, via inhalation, or intravenous administration. In some embodiments, the fumarate (e.g., DMF, MMF, or combinations thereof) is administered orally in any of the methods described above.

The time period for which the subject is dosed with the fumarate (e.g., DMF, MMF, or combinations thereof) in any of the methods described above can range, for example, from about 1 week to the remaining lifespan of the subject. The fumarate (e.g., DMF, MMF, or combinations thereof) and its composition can be dosed, for example, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 20 weeks, at least 30 weeks, at least 40 weeks, at least 50 weeks, at least 1 year, at least 60 weeks, at least 70 weeks, at least 80 weeks, at least 90 weeks, at least 100 weeks, at least 2 years, at least 3 years, at least 4 years, at least 5 years, at least 6 years, at least 7 years, at least 8 years, at least 9 years, at least 10 years, at least 20 years, at least 30 years, at least 40 years, at least 50 years, at least 60 years, at least 70 years, at least 80 years, at least 90 years, or at least 100 years. In one embodiment, the fumarate (e.g., DMF, MMF, or combinations thereof) and its composition can be dosed, for example, for a period of time ranging from about 1 week to about 100 years, about 1 week to about 90 years, about 1 week to about 80 years, about 1 week to about 70 years, about 1 week to about 60 years, about 1 week to about 50 years, about 1 week to about 40 years, about 1 week to about 30 years, about 1 week to about 20 years, about 1 week to about 10 years, about 1 week to about 9 years, about 1 week to about 8 years, about 1 week to about 7 years, about 1 week to about 6 years, about 1 week to about 5 years, about 1 week to about 4 years, about 1 week to about 3 years, about 1 week to about 2 years, about 1 week to about 100 weeks, about 1 week to about 1 year, about 1 week to about 50 weeks, about 1 week to about 40 weeks, about 1 week to about 30 weeks, about 1 week to about 20 weeks, about 1 week to about 10 weeks, or about 1 week to about 5 weeks. In another embodiment, the fumarate (e.g., DMF, MMF, or combinations thereof) and its composition can be dosed, for example, about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 1 year, about 60 weeks, about 70 weeks, about 80 weeks, about 90 weeks, about 100 weeks, about 2 years, about 3 years, about 4 years, about 5 years, about 6 years, about 7 years, about 8 years, about 9 years, about 10 years, about 20 years, about 30 years, about 40 years, about 50 years, about 60 years, about 70 years, about 80 years, about 90 years, or about 100 years.

In one embodiment of the methods described above, administration of the fumarate (e.g., DMF, MMF, or combinations thereof) or composition containing the fumarate (e.g., DMF, MMF, or combinations thereof) to a subject or group of subjects results in reducing the frequency of relapse in the subject relative to subject or group of subjects treated with placebo.

“Reduction in the frequency of relapse” means that the number of relapses in a treated subject or a group of treated subjects are decreased relative to the number of relapses in a subject or a group of subjects treated with placebo. For example, a 50% reduction in frequency of relapse means that the group of treated subjects had on average 50% fewer relapses than the placebo group.

The reduction of the frequency of relapse in a subject or a group of treated subjects can range, for example, from about 10% to about 90% after at least 1 year of treatment. In one embodiment, the frequency of relapse in a subject or a group of treated subjects can range, for example, from about 10% to about 90% after at least 2 years of treatment.

The frequency of relapse can be reduced, for example, from a range of about 10% to 100% after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment). The frequency of relapse can be reduced, for example, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment). The frequency of relapse can be reduced, for example from about 10% to about 90%, about 10% to about 80%, about 10% to about 70%, about 10% to about 60%, about 20% to about 90%, about 30% to about 90%, about 40% to about 90%, about 50% to about 90%, about 60% to about 90%, about 70% to about 90%, about 20% to about 70%, or about 25% to about 65% after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment).

The reduction of the frequency of relapse in a subject or a group of subjects treated with fumarate (e.g., DMF, MMF, or combinations thereof) can be, for example, from about 10% to about 90% after at least 1 year of treatment. In one embodiment, the reduction in the proportion of subjects or group of subjects treated with fumarate (e.g., DMF, MMF, or combinations thereof) relapsing is, for example, from about 10% to about 90% after at least 2 years of treatment.

The reduction in the proportion of subjects or group of subjects treated with fumarate (e.g., DMF, MMF, or combinations thereof) relapsing is, for example, from a range of about 10% to 90% after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment). The reduction in the proportion of subjects or group of subjects treated with fumarate (e.g., DMF, MMF, or combinations thereof) relapsing is, for example, at least 30% at least 30%, at least 35%, at least 40%, at least 45%, at least 50%, at least 55%, at least 60%, at least 65%, at least 70%, at least 75%, or at least 80% after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment). The reduction in the proportion of subjects or group of subjects treated with fumarate (e.g., DMF, MMF, or combinations thereof), for example from about 10% to about 90%, about 10% to about 80%, about 10% to about 70%, about 10% to about 60%, about 20% to about 90%, about 30% to about 90%, about 40% to about 90%, about 20% to about 80%, about 30% to about 80%, or about 40% to about 80% after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment)

In one embodiment, administration of the fumarate (e.g., DMF, MMF, or combinations thereof) or its composition in any of the described methods results in reducing the probability of relapse in a treated subject or a group of treated subjects relative to a subject or group of subjects treated with placebo.

“Reduction in the probability of relapse” is the difference, at the same time point, between the probability of relapse of a subject or a group of subjects treated with placebo and the probability of relapse in a treated subject or a group of treated subjects. Probability data can be obtained from Kaplan-Meier plots of probability of relapse that have cumulative probability of relapse on the plot ordinate and time on the plot abscissa.

Reduction in the probability of relapse in the subject or group of subjects can, for example, be: at least 0.005 after any time period of treatment, at least 0.01 after any time period of treatment, at least 0.1 at any time period of treatment, at least 0.05 after at least 12 weeks of treatment, at least 0.06 after at least 24 weeks of treatment, at least 0.14 after at least 36 weeks of treatment, at least 0.20 after at least 48 weeks of treatment, or at least 0.30 after at least 60 weeks of treatment.

Reduction in the probability of relapse can be, for example, at least 0.005, at least 0.01, at least 0.05, at least 0.1, at least 0.15, at least 0.2, at least 0.25, at least 0.3, at least 0.35, at least 0.4, at least 0.45, at least 0.5, or at least 0.55, after any time period of treatment (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment). For example, the probability of relapse can be about 0.01 to about 0.90, about 0.01 to about 0.80, about 0.01 to about 0.70, about 0.01 to about 0.60, about 0.01 to about 0.50, about 0.01 to about 0.40, about 0.10 to about to about 0.30, about 0.01 to about 0.20, or about 0.01 to about 0.10 after any time period of treatment (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment). In one embodiment, the reduction in probability of relapse in any subject or group of subjects described above after 5, 12, 24, 36, 48, 60, 96, or 100 weeks or 1, 2, 3, 4, 5, 10, 15, or 20 years of treatment is at least 0.005.

In one embodiment, administration of the fumarate (e.g., DMF, MMF, or combinations thereof) or its composition in any method described above results in reducing the annualized relapse rate in a treated subject or group of treated subjects relative to a subject or group of subjects treated with placebo.

In one embodiment, the annualized relapse rate in the subject or group of subjects can be reduced by, for example: at least 30%, about 30% to about 70%, about 50%, at least 50%, about 45% to about 55%, about 53%, about 48%, about 30% after any time period of treatment e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment).

The reduction in annualized relapse rate can range from about 1% to 100%. For example, the reduction in annualized relapse rate can be at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% after any time period of treatment (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment).

The reduction in annualized relapse rate in any subject or group of subjects described above can be, for example, about 1% to about 90%, about 1% to about 80%, about 1% to about 70%, about 1% to about 60%, about 1% to about 50%, about 1% to about 40%, about 1% to about 30%, about 1% to about 20%, about 1% to about 10%, about 10% to about 80%, about 10% to about 70%, about 20% to about 70%, about 25% to about 75%, about 20% to about 80%, about 30% to about 70%, about 30% to about 80%, about 35% to about 65%, about 40% to about 60%, or about 45% to about 55% after any time period of treatment (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment).

The reduction in annualized relapse rate in any subject or group of subjects described above can, for example, change from about 25% after being treated for about 24 weeks to about 50% after being treated for about 2 years. In one embodiment, the reduction in annualized relapse rate in any subject or group of subjects described above can, for example, change from about 30% after being treated for about 24 weeks to about 45% after being treated for about 2 years or about 40% after being treated for about 24 weeks to about 50% after being treated for about 2 years. In one embodiment, the subject or group of subjects is administered 480 mg or 720 mg fumarate (e.g., DMF, MMF, or combinations thereof) per day.

In one embodiment, the reduction in annualized relapse rate in the subject or group of subjects decreases about 10% every 2 years over a treatment period of about 4 years. In one embodiment, the reduction in annualized relapse rate in the subject or group of subjects decreases about 10% every 2 years over a treatment period of about 4 years when the subject is administered about 480 mg of a fumarate daily (e.g., DMF, MMF, or combinations thereof daily).

In one embodiment, administration of the fumarate (e.g., DMF, MMF, or combinations thereof) or its composition in any of the methods described above results in reduction of the risk of disability progression in a treated subject or group of treated subjects relative to a subject or group of subjects treated with placebo.

Disability progression is measured by EDSS. The risk of disability progression in any subject or group of subjects described above can be reduced by, for example: about 30% to about 40% at about 100 weeks of treatment, about 31% to about 37% at about 50 weeks of treatment, about 30% to about 40% at about 50 weeks of treatment, or about 31% to about 37% at about 100 weeks of treatment.

The risk of disability progression in any subject or group of subjects described above can be reduced from about 1% to 100% after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment). For example, the risk of disability progression can be reduced by at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% after any time period of treatment (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment).

The risk of disability progression can be reduced, for example, from about 1% to about 90%, about 1% to about 80%, about 1% to about 70%, about 1% to about 60%, about 1% to about 50%, about 1% to about 40%, about 1% to about 30%, about 1% to about 20%, about 1% to about 10%, about 20% to about 70%, about 25% to about 65%, about 30% to about 60%, about 35% to about 55%, or about 40% to about 50% after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment).

In one embodiment, administration of the fumarate (e.g., DMF, MMF, or combinations thereof) or its composition in any of the methods described above results in reduction of the number of new or newly enlarging T2 lesions in a treated subject or group of treated subjects relative to a subject or group of treated subjects treated with placebo. The reduction can be ascertained by routine magnetic resonance imaging (“MRI”) methods.

The number of new or newly enlarging T2 lesions in any subject or group of subjects described above can be reduced by, for example: about 70% to about 90% after at least 100 weeks of treatment, at least 85% after at least 100 weeks of treatment, or at least 74% after at least 100 weeks of treatment.

The reduction in the number of new or newly enlarging T2 lesions in any subject or group of subjects described above can range from about 1% to 100% after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment). For example, the reduction in the number of new or newly enlarging T2 lesions can be at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment).

The reduction in the number of new or newly enlarging T2 lesions in any subject or group of subjects described above can range, for example, from about 1% to about 90%, about 1% to about 80%, about 1% to about 70%, about 1% to about 60%, about 1% to about 50%, about 1% to about 40%, about 1% to about 30%, about 1% to about 20%, about 1% to about 10%, about 30% to about 99%, about 35% to about 99%, about 40% to about 99%, about 45% to about 99%, about 50% to about 99%, about 30% to about 95%, about 35% to about 95%, about 40% to about 95%, about 45% to about 95%, about 50% to about 95%, about 30% to about 90%, about 35% to about 90%, about 40% to about 90%, about 45% to about 90%, about 50% to about 90%, about 30% to about 85%, about 35% to about 85%, about 40% to about 85%, about 45% to about 85%, about 50% to about 85%, about 30% to about 80%, about 35% to about 80%, about 40% to about 80%, about 45% to about 80%, or about 50% to about 80%, about 60% to about 100%, about 65% to about 95%, about 70% to about 95%, about 65% to about 90%, about 65% to about 85%, or about 65% to about 80% after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment).

In one embodiment, administration of the fumarate (e.g., DMF, MMF, or combinations thereof) or its composition in any of the methods described above results in reduction of the number of new non-enhancing T1 hypointense lesions in a treated subject or group of treated subjects relative to a subject or group of subjects treated with placebo. The reduction can be ascertained by routine magnetic resonance imaging (“MRI”) methods. The reduction of the number of new non-enhancing T1 hypointense lesions in any subject or group of subjects described above can be determined after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment).

The reduction of the number of new non-enhancing T1 hypointense lesions in any subject or group of subjects described above can range, for example, from about 30% to about 90%, about 35% to about 90%, about 40% to about 90%, about 45% to about 90%, about 50% to about 90%, about 30% to about 85%, about 35% to about 85%, about 40% to about 85%, about 45% to about 85%, about 50% to about 85%, about 30% to about 80%, about 35% to about 80%, about 40% to about 80%, about 45% to about 80%, or about 50% to about 80% after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment).

In one embodiment, the number of new non-enhancing T1 hypointense lesions in any subject or group of subjects described above can be reduced by, for example, about 50% to about 70% after at least 48 weeks of treatment. In another embodiment, the number of new non-enhancing T1 hypointense lesions in any subject or group of subjects described above can be reduced by, for example, at least 60% after at least 96 weeks of treatment. In yet another embodiment, the number of new non-enhancing T1 hypointense lesions in any subject or group of subjects described above can be reduced by, for example, about 55% to about 65% after at least 48 weeks of treatment. In still yet another embodiment, the number of new non-enhancing T1 hypointense lesions in any subject or group of subjects described above can be reduced by, for example, about 60% to about 70% after at least 96 weeks of treatment.

In one embodiment, administration of the fumarate (e.g., DMF, MMF, or combinations thereof) or its composition in any of the above described methods results in reduction in the number of Gd+ lesions in the treated subject or group of treated subjects relative to a subject or group of treated subjects treated with placebo. The reduction in the number of Gd+ lesions in any subject or group of subjects described above can be determined after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment).

The percentage reduction of the number of Gd+ lesions in any subject or group of subjects described above can range, for example, from about 10% to 100% after, for example, at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment.

The percentage reduction in the number of Gd+ lesions in any subject or group of subjects described above can range, for example, from about 10%, to about 98%, about 10% to about 97%, about 10% to about 96%, about 10% to about 95%, about 10% to about 94%, about 10% to about 93%, about 10% to about 92%, about 10% to about 91%, about 10% to about 90%, about 10% to about 85%, about 10% to about 80%, about 10% to about 75%, about 10% to about 70%, about 15% to about 99%, about 20% to about 99%, about 25% to about 99%, about 30% to about 99%, about 35% to about 99%, about 40% to about 99%, about 45% to about 99%, about 50% to about 99%, about 55% to about 99%, about 60% to about 99%, about 65% to about 99%, or about 70% to about 99%, about 30% to about 95%, about 35% to about 95%, about 40% to about 95%, about 45% to about 95%, about 50% to about 95%, about 30% to about 90%, about 35% to about 90%, about 40% to about 90%, about 45% to about 90%, about 50% to about 90%, about 30% to about 85%, about 35% to about 85%, about 40% to about 85%, about 45% to about 85%, about 50% to about 85%, about 30% to about 80%, about 35% to about 80%, about 40% to about 80%, about 45% to about 80%, or about 50% to about 80% after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment).

The percentage reduction in the number of Gd+ lesions in any subject or group of subjects described above can be, for example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment).

The percentage reduction in the number of Gd+ lesion in any subject or group of subjects described above can be, for example, about 20%, about 25%, about 30%, about 35%, about 40%, about 45%, about 50%, about 55%, about 60%, about 65%, about 70%, about 75%, about 80%, or about 90% after any time period of administration (e.g., at least 5, 12, 24, 36, 48, 60, 96, or 100 weeks or 1, 2, 3, 4, 5, 10, 15, or 20 years of treatment or about 5, 12, 24, 36, 48, 60, 96, or 100 weeks or 1, 2, 3, 4, 5, 10, 15, or 20 years of treatment).

The following examples are illustrative and do not limit the scope of the disclosure of the claims.

### EXAMPLES

### Example 1

### Study Parameters and Adverse Events of a First Phase 3 Clinical Trial

A randomized, double-blind, placebo-controlled, multicenter, phase 3 clinical trial was conducted to evaluate the efficacy and safety of dimethyl fumarate over 2 years in subjects with relapsing-remitting multiple sclerosis (“RR-MS”).

Subjects 18-55 years of age with McDonald criteria diagnosis of RR-MS and an Expanded Disability Status Scale score of 0.0-5.0 (inclusive) were eligible for enrolment. Subjects were randomly assigned in a 1:1:1 ratio to placebo, dimethyl fumarate 240 mg PO twice daily (BID), or dimethyl fumarate three times daily (TID). Safety and tolerability were assessed by continuous adverse event (“AE”) monitoring and laboratory tests at all monthly visits. Additionally, physical examination, vital signs, and 12-lead ECG were evaluated. As an ethical consideration, subjects' reconsent was required following any protocol-defined disability progression or relapse.

Of the 1234 subjects dosed with placebo (n=408), dimethyl fumarate BID (n=410), or dimethyl fumarate TID (n=416) approximately 23% of subjects withdrew from the study in each treatment arm. AEs were reported by 95%, 96%, and 95%, of subjects receiving placebo, dimethyl fumarate BID, and dimethyl fumarate TID, respectively. AEs resulted in 55 (13%), 65 (16%), and 68 (16%) subjects discontinuing treatment in the placebo, dimethyl fumarate BID, and dimethyl fumarate TID arms, respectively. The most frequently reported AEs were flushing, MS relapse, nasopharyngitis, headache, diarrhea, and fatigue. Subjects reported flushing more frequently in the dimethyl fumarate arms (5%, 38%, and 32% of subjects receiving placebo, dimethyl fumarate BID, and dimethyl fumarate TID, respectively) and MS relapse was reported more frequently in the placebo arm (46% versus 27% for dimethyl fumarate BID or TID). Severe AEs were reported in 17%, 16%, and 17% of subjects receiving placebo, dimethyl fumarate BID, and dimethyl fumarate TID, respectively. Incidence of infections and infestations (64%-68%) and severe infections and infestations (2-3%) was similar in all treatment groups; no opportunistic infections were observed in the dimethyl fumarate treatment arms. One death, in the dimethyl fumarate TID arm, was the result of a motor vehicle accident.

### Example 2

### Efficacy Data Regarding Relapse and Disability

The primary endpoint of the study in Example 1 was the proportion of subjects relapsing at 2 years, with relapses confirmed by an independent neurology evaluation committee (“INEC”) to ensure consistent and accurate reporting across sites. Secondary clinical efficacy endpoints at 2 years were the annualized relapse rate (“ARR”) and disability progression using EDSS. Efficacy analyses were conducted on the intention-to-treat population.

All primary and secondary endpoints of the study were met. Dimethyl fumarate BID and TID reduced the proportion of subjects relapsing by 49% and 50%, respectively, compared with placebo (P<0.0001) at 2 years. ARR was 0.36 with placebo, and 0.17 and 0.19 with dimethyl fumarate BID and TID, corresponding to reductions of 53% and 48% for dimethyl fumarate BID and TID (P<0.001). The risk of confirmed, 12-week disability progression was reduced by 38% with dimethyl fumarate BID (P<0.01) and approximately 34% with dimethyl fumarate TID (P<0.05). The overall incidence of adverse and serious adverse events was similar among the placebo and both dimethyl fumarate treatment groups.

The results from this large phase 3 study support the potential of dimethyl fumarate as an effective oral treatment for subjects with RR-MS.

### Example 3

### Efficacy Data Regarding Lesion Loads

Subjects from 76 sites out of 198 participating in the study described in Example 1 had MRI scans at baseline, 24 weeks, 1 year and 2 years. Number of new or newly enlarging T2 lesions and number of gadolinium-enhancing (Gd+) lesions at 2 years were secondary endpoints of the study. Analyses were conducted on the MRI intention-to-treat population.

The mean number of new or newly enlarging T2 lesions was reduced by 85% and 74% in the dimethyl fumarate BID and TID groups, respectively (P<0.001 for both) and the mean number of Gd+ lesions was reduced by 94% and 72%, respectively (P<0.001 for both) in the BID and TID groups, compared with placebo.

The results of the MRI analysis of the study described in Example 1 demonstrate a potent anti-inflammatory effect on focal white matter lesions, in line with that of the most potent approved agents, and support the clinical findings and potential of dimethyl fumarate as an effective oral treatment for subjects with RR-MS.

### Example 4

### Quality of Life in Subjects

Subjects participating in the study described in Example 1 were provided an SF-36 questionnaire to measure the subjects' health status and health-related quality of life (“QoL”) at baseline, 6, 12, and 24 months, on eight multi-item, 100-point scales, with higher scores indicating higher QoL. These scores were used to calculate the Physical Component Summary (“PCS”) and Mental Component Summary (“MCS”) scores. In addition, the subjects' global impression of well-being was assessed at baseline and every 3 months using a 100-point, visual analogue scale (VAS) with higher score indicating improvement in well-being.

Compared with placebo, mean SF-36 PCS scores at 2 years favored dimethyl fumarate BID and TID (placebo [42.0] versus dimethyl fumarate BID [43.4] and TID [44.2]) with a significant difference in PCS change over the 2 years (P<0.001 for both) indicating subjects felt physically better with dimethyl fumarate treatment. Similar trends were observed in scores on the SF-36 Mental Component Scale (placebo [44.6] versus dimethyl fumarate BID [45.5] and TID [46.1]; P=0.065, and P<0.002, respectively). Compared with mean VAS score in the placebo arm (60.3) dimethyl fumarate BID and TID scores (64.3 and 65.7) indicated improvement in well-being, with a significant difference in VAS score (P=0.003 and P<0.001) at 2 years.

Based on subject reported measures, dimethyl fumarate significantly improved physical functioning and general well-being in subjects with RR-MS. Dimethyl fumarate benefits on relapse rate and EDSS progression in the study described in Example 1 are reflected in subject-reported, health-related quality of life, further supporting its role as an effective oral treatment option for subjects with RR-MS.

### Example 5

### Efficacy Data Regarding Preserving/Increasing Myelin Content

The study described in Example 1 included a substudy to examine change in magnetization transfer ratio (“MTR”) with treatment. MTR imaging has been proposed for use as a biomarker of changes in the myelin content of brain white matter. The aim of the study was to determine change in brain and lesion MTR in RR-MS. MTR scans were obtained in a subset of subjects at baseline and 6, 12 and 24 months, using manufacturer-supplied MT pulse sequences.

MTR substudies were implemented at 64 of 76 MRI sites (84%) and included a total of 540 subjects: 176 in the dimethyl fumarate 240 mg bid group, 184 in the dimethyl fumarate 240 mg tid group, and 180 in the placebo group. In the dimethyl fumarate 240 mg bid and tid groups, larger increases from baseline were observed in whole brain MTR (0.129% and 0.096%), respectively) and normal appearing brain tissue MTR (0.190%, 0.115%, respectively) compared with placebo (−0.386% and −0.392% for whole brain and normal appearing brain MTRs, respectively). These results were maintained even when data was restricted to those generated by subjects who did not experience relapses. The results from this study demonstrate that dimethyl fumarate preserves and/or increases myelin content in subjects with RR-MS.

Additionally, as shown in Tables 1 and 2 below, dimethyl fumarate 240 mg BID and dimethyl fumarate 240 mg BID were more efficacious in subjects under 40 years of age than subjects that were 40 years or older.

TABLE 1
Dimethyl fumarate 240 mg BID

% Reduction vs.
Placebo

| Endpoint | <40 years | ≧40 years |
|---|---|---|
| Proportion of patients | 59 | 26 |
| relapsed at 2 years | | |
| Annualized relapse rate at 2 | 63 | 34 |
| years | | |
| Time to confirmed disability | 62 | 8 |
| progression at 2 years | | |

TABLE 2
Dimethyl fumarate 240 mg TID

% Reduction vs.
Placebo

| Endpoint | <40 years | ≧40 years |
|---|---|---|
| Proportion of patients | 56 | 37 |
| relapsed at 2 years | | |
| Annualized relapse rate at 2 | 59 | 29 |
| years | | |
| Time to confirmed disability | 38 | 32 |
| progression at 2 years | | |

### Example 7

### Second Phase 3 Clinical Trial

A second randomized, double-blind, placebo-controlled, multicenter, phase 3 clinical trial was conducted to evaluate the efficacy and safety of dimethyl fumarate over 2 years in subjects with relapsing-remitting multiple sclerosis (“RR-MS”).

Subjects 18-55 years of age with RR-MS and an Expanded Disability Status Scale score of 0.0-5.0 (inclusive) and having one or more relapses in the prior 12 months or one or more Gd⁺ lesions in the prior 6 weeks were eligible for enrolment. Subjects were randomly assigned to placebo, dimethyl fumarate 240 mg PO twice daily (BID), dimethyl fumarate three times daily (TID), glatiramer acetate 20 mg SC once daily (QD).

Of the 1417 total subjects dosed with placebo (n=363), dimethyl fumarate BID (n=359), dimethyl fumarate TID (n=350) 23%, 21%, 21%, and 17% of subjects withdrew from the study in each treatment arm, respectively. AEs were reported by 92%, 94%, 92%, and 87% of subjects receiving placebo, dimethyl fumarate BID, dimethyl fumarate TID, and glatiramer acetate respectively. AEs resulted in 38 (10%), 44 (12%), 41 (12%), and 35 (10%) subjects discontinuing treatment in the placebo, dimethyl fumarate BID, dimethyl fumarate TID, and glatiramer acetate arms, respectively. The most frequently reported AEs associated with dimethyl fumarate were flushing, diarrhea, nausea, upper respiratory tract infection, abdominal pain, and proteinuria.

The primary endpoint of the second phase 3 clinical trial was the annualized relapse rate over 2 years.

Secondary endpoints of the second phase 3 clinical trial were the number of new or newly enlarging T2 hyperintense lesions at 2 years, proportion of patients relapsed at 2 years, disability progression as measured by EDSS, and number of new T1 hypointense lesions at 2 years.

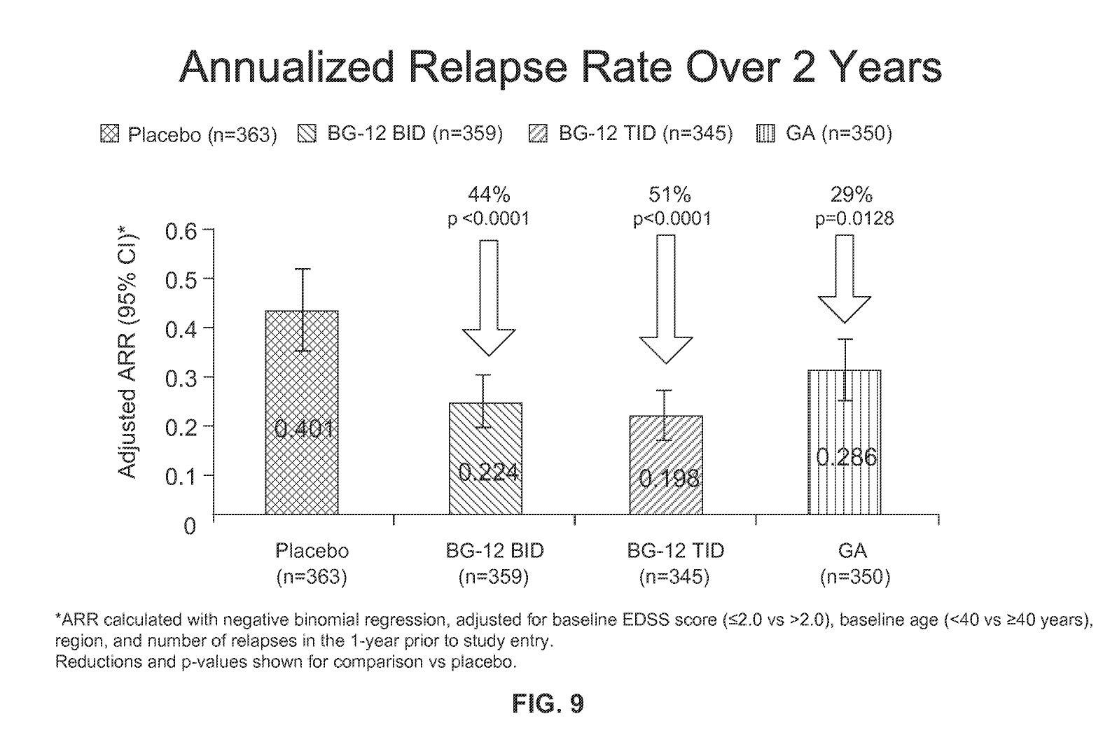

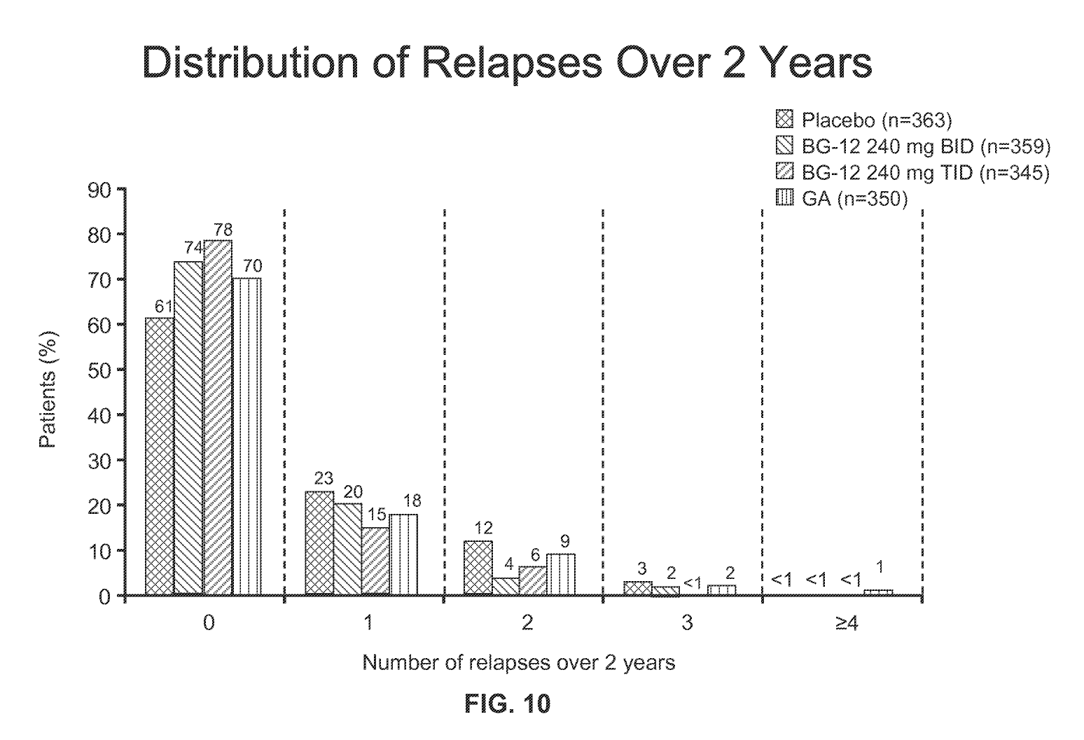

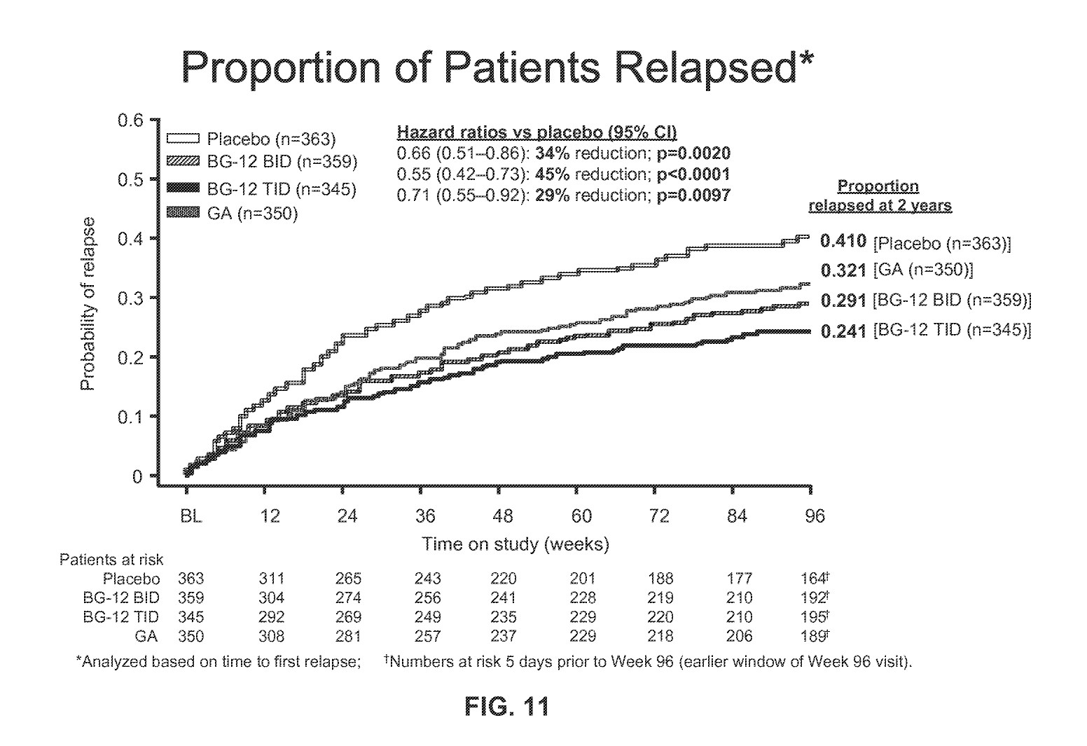

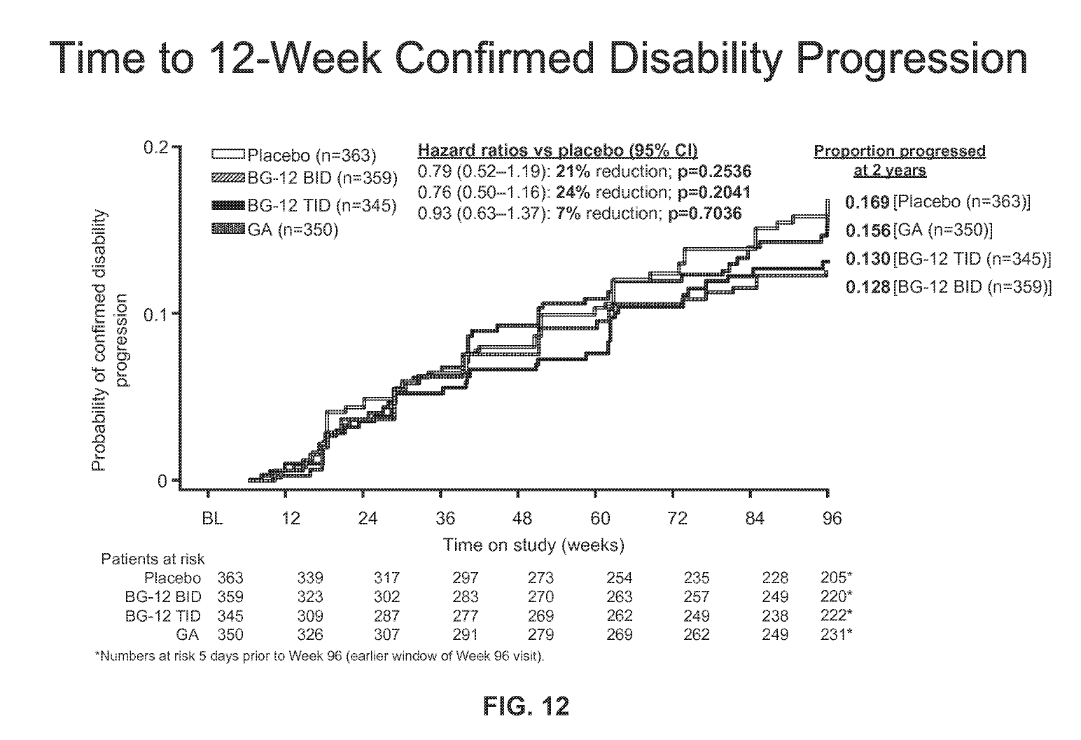

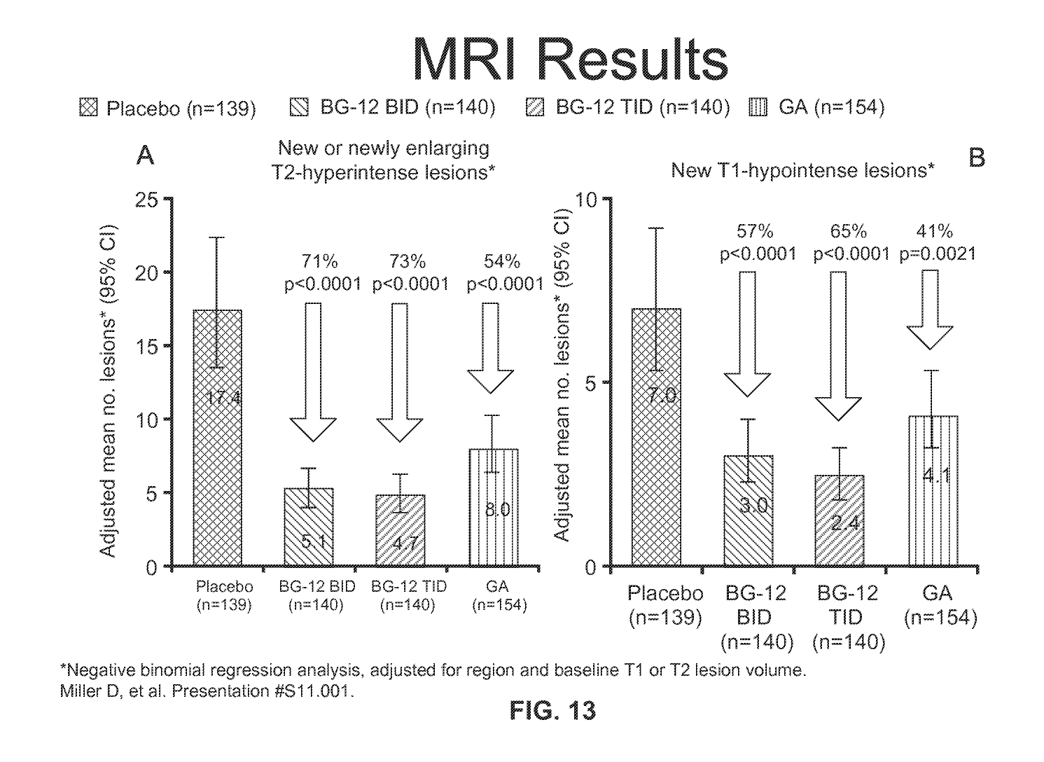

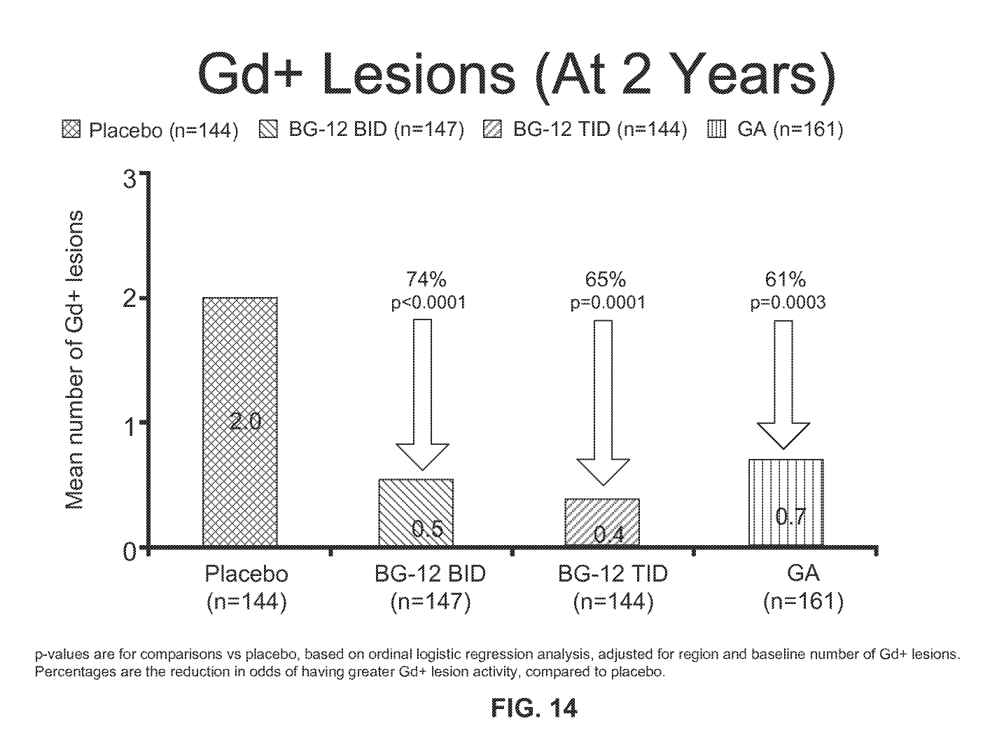

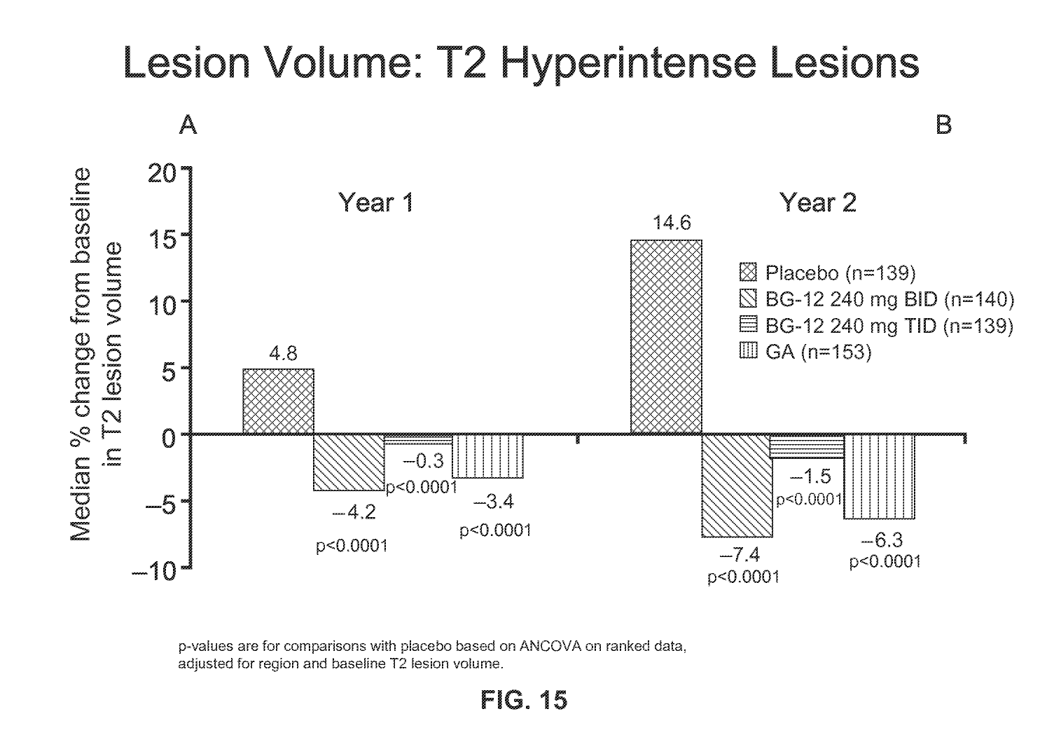

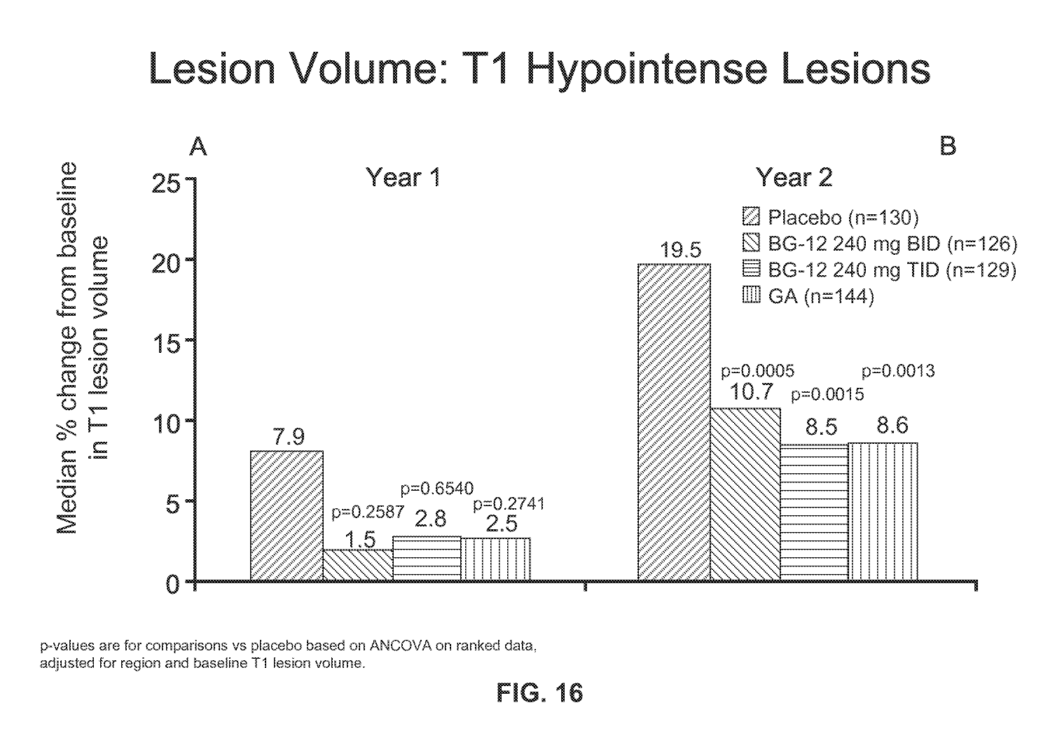

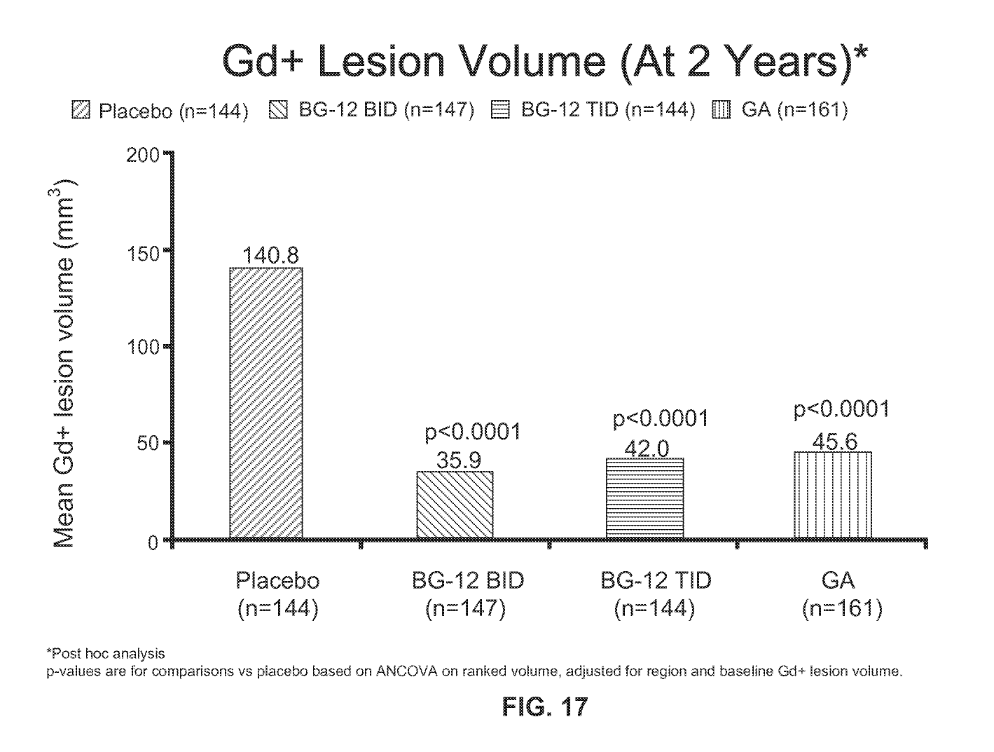

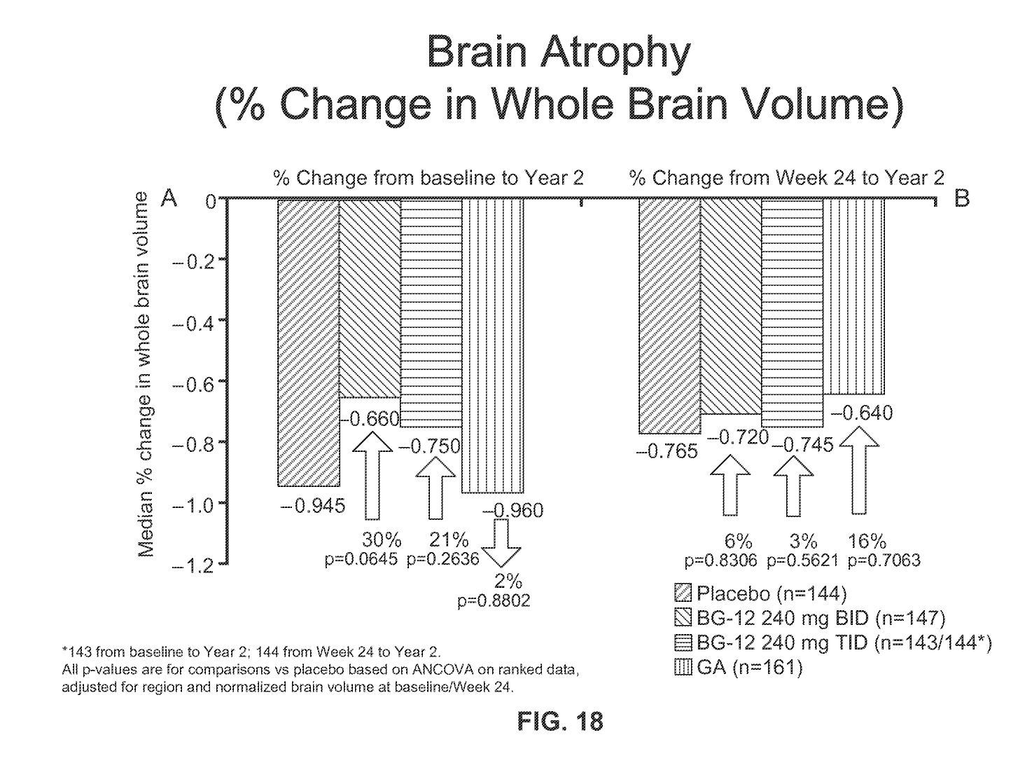

Results of the second phase 3 clinical trial are shown in FIGS. 9-18.

All publications, patents, and patent applications mentioned in this application are herein incorporated by reference to the same extent as if each individual publication, patent, or patent application was specifically and individually indicated to be incorporated by reference.

1. A method of treating a subject having multiple sclerosis, comprising orally administering to the subject about 480 mg per day of a fumarate for a period of time sufficient to achieve one or more of the following changes:
(a) reduced frequency of relapse in the subject;
(b) reduced probability of relapse in the subject;
(c) reduced annualized relapse rate in the subject;
(d) reduced risk of disability progression in the subject;
(e) reduced number of new or newly enlarging T2 lesions in the subject;
(f) reduced number of new non-enhancing T1 hypointense lesions in the subject; and
(g) reduced number of Gd+ lesions in the subject; wherein the changes (a)-(g) are relative to a subject receiving placebo.

2. The method of claim 1, wherein the fumarate is dimethyl fumarate.

3. The method of claim 1, wherein the fumarate is a compound that is converted to methyl hydrogen fumarate in vivo.

4. The method of claim 1, wherein the multiple sclerosis is relapsing-remitting multiple sclerosis.

5. The method of claim 1, wherein the change is a reduced frequency of relapse in the subject.

6-7. (canceled)

8. The method of claim 1, wherein the change is a reduced probability of relapse in the subject.

9-10. (canceled)

11. The method of claim 1, wherein the change is a reduced annualized relapse rate in the subject.

12-13. (canceled)

14. The method of claim 1, wherein the change is a reduced risk of disability progression in the subject.

15. (canceled)

16. The method of claim 1, wherein the change is a reduced number of new or newly enlarging T2 lesions in the subject.

17. (canceled)

18. The method of claim 1, wherein the change is a reduced number of new non-enhancing T1 lesions in the subject.

19. (canceled)

20. The method of claim 1, wherein the change is a reduced number of Gd+ lesions in the subject.

21-27. (canceled)

28. The method of claim 1, wherein the fumarate is dimethyl fumarate and is administered daily as two doses with each dose being about 240 mg of dimethyl fumarate.

29-30. (canceled)

31. The method of claim 1, wherein the fumarate is dimethyl fumarate and is administered for at least 2 years.

32. A method of treating a subject having relapsing-remitting multiple sclerosis, comprising orally administering to the subject about 480 mg per day of a fumarate for a period of time sufficient to achieve one or more of the following changes:
(a) reduced annualized relapse rate of at least 40%;
(b) reduced risk of disability progression of at least 30%; and
(c) reduced number of new or newly enlarging T2 lesions of at least 70%;
wherein
the changes (a)-(c) are relative to a subject receiving placebo.

33-35. (canceled)

36. The method of claim 32, wherein the fumarate is dimethyl fumarate and is administered for at least 2 years.

37. The method of claim 36, wherein the fumarate is administered daily as two doses with each dose comprising about 240 mg of dimethyl fumarate.

38. A method of treating a subject having relapsing-remitting multiple sclerosis, comprising orally administering to the subject a composition comprising an excipient and a dose of a fumarate for at least 24 weeks, wherein the fumarate is the only active ingredient in the composition and the therapeutically effective dose is about 480 mg per day.

39. The method of claim 38, wherein a reduction in annualized relapse rate in the subject changes from about 30% after being treated for about 24 weeks to about 45% after being treated for about 2 years, and wherein the reduction in annualized relapse rate is relative to a subject receiving placebo.

40. The method of claim 38, wherein a reduction in annualized relapse rate in the subject changes from about 40% after being treated for about 24 weeks to about 50% after being treated for about 2 years, and wherein the reduction in annualized relapse rate is relative to a subject receiving placebo.

41. A method of preserving and/or increasing myelin content in a subject having multiple sclerosis comprising administering about 480 mg per day of a fumarate to the subject for a period of time sufficient to preserve and/or increase the myelin content.

42. The method of claim 41, wherein the fumarate is dimethyl fumarate and is administered daily as two doses with each dose being about 240 mg of dimethyl fumarate.
